(19) United States
(12) Patent Application Publication
(10) Pub. No.: US 2006/0222655 A1
(43) Pub. Date: Oct. 5, 2006

(54) Compositions and methods for preventing and treating endotoxin-related diseases and conditions

(76) Inventor: James McShane, Wake Forest, NC (US)

Correspondence Address: CLARK & ELBING LLP, 101 FEDERAL STREET, BOSTON, MA, 02110, US

(21) Appl. No.: 10/547,599

(22) PCT Filed: Mar. 5, 2004
(86) PCT No.: PCT/US04/06713

§ 371 Date: Sep. 1, 2005

Related U.S. Application Data
(60) Provisional application No. 60/452,022, filed on Mar. 5, 2003.

(51) Int. Cl. A61K 31/739; A61K 39/00

(52) U.S. Cl. 424/184.1; 514/54

(57) ABSTRACT

The invention provides pharmaceutical compositions for preventing and treating endotoxin-related diseases and conditions, as well as methods for making and using such compositions.

### BACKGROUND OF THE INVENTION

This invention relates to compositions and methods for preventing and treating endotoxin-related diseases and conditions.

Since the 1930's, the increasing use of immunosuppressive therapy and invasive devices, as well as the increased incidence of antibiotic resistance in bacteria, have led to a gradual rise in the occurrence of sepsis and septic shock. Currently, the estimated incidences in the United States of sepsis and septic shock are 400,000 and 200,000 patients/year, respectively. This results in about 100,000 fatalities/year, making septic shock the most common non-coronary cause of death in the hospital Intensive Care Unit (ICU). Currently, ICU therapy for septic shock generally involves treatment with antibiotics, cardiovascular resuscitation, vasopressor/ionotrope therapy, and/or ventilatory support. This ICU care can cost up to $1,500/day/patient, resulting in an average total cost per patient of $13,000 to $30,000, due to the typical length of ICU stay.

Sepsis and septic shock are caused by the release of a molecule known as endotoxin or lipopolysaccharide (LPS) from the walls of growing and dying gram-negative bacteria. The released endotoxin induces many pathophysiological events, such as fever, shock, disseminated intravascular coagulation (DIC), and hypotension, in infected patients. Medicines for the treatment of gram-negative sepsis have been desired for some time, especially drugs that block endotoxin or cytokines induced by endotoxin-mediated cellular stimulation. To this end, various strategies for treatment have included administration of antibodies or other agents against LPS, or cytokines, such as TNF-α and interleukin-1. For various reasons, these approaches have failed.

While endotoxin itself is a highly heterogenous molecule, the toxic properties of endotoxin are attributable to the highly conserved hydrophobic lipid A portion of the molecule. An effective drug that acts as an antagonist to this conserved structure is known as E5564 (also known as compound 1287 and SGEA). This drug is described as compound 1 in U.S. Pat. No. 5,935,938, which is incorporated herein by reference.

### SUMMARY OF THE INVENTION

The invention provides compositions including the antiendotoxin compound E5564, which has the formula: 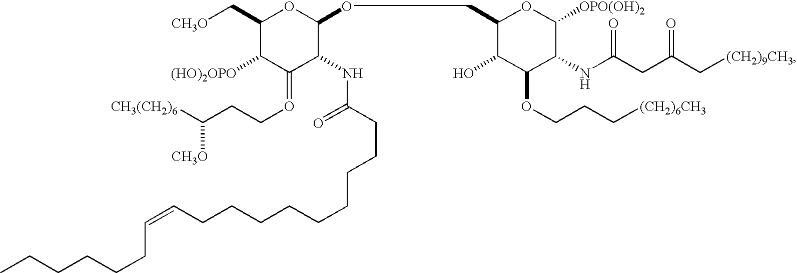
pharmaceutically acceptable salts thereof, and an antioxidant.

Examples of antioxidants that can be used in the compositions of the invention include butylated hydroxyanisole (BHA), butylated hydroxytoluene (BHT), propyl gallate, sodium sulfite, sodium thiosulfate, monothioglycerol, tert-butyl hydroquinone, ethoxyquin, dithiothreitol, and derivatives thereof. The compositions of the invention can also include disaccharide stabilizing agents (e.g., a disaccharide such as lactose, sucrose, trehalose, or maltose) and/or include sodium ions in amounts of 0.5-10 mM or ≦2 mM, so as to stabilize the micelle size of the antiendotoxin compound at about 7-9 m during lyophilization.

The invention also provides methods of making pharmaceutical compositions including antiendotoxin compounds, which involve admixing the compounds with an antioxidant. An example of an antiendotoxin compound that can be included in these compositions is E5564 (or pharmaceutically acceptable salts thereof). Examples of antioxidants present in these compositions include butylated hydroxyanisole, butylated hydroxytoluene, propyl gallate, sodium sulfite, sodium thiosulfate, monothioglycerol, tert-butyl hydroquinone, ethoxyquin, dithiothreitol, and derivatives thereof.

Also included in the invention are methods of making pharmaceutical compositions including antiendotoxin compounds. These methods can include the steps of: (i) dissolving the antiendotoxin compound in an aqueous solution of sodium hydroxide; (ii) adding a disaccharide stabilizer (e.g., lactose) to the solution; (iii) adding an antioxidant (e.g., butylated hydroxyanisole, butylated hydroxytoluene, propyl gallate, sodium sulfite, sodium thiosulfate, monothioglycerol, tert-butyl hydroquinone, ethoxyquin, dithiothreitol, or a derivative thereof) to the solution; (iv) lowering the pH of the solution (to, e.g., about pH 7-8, by use of, e.g., a phosphoric acid solution); (v) filter sterilizing the solution; and (vi) freeze-drying the solution (using, e.g., a process including the use of a shelf temperature of 0° C.-20° C.).

The invention also includes methods of preventing or treating endotoxemia in patients, involving administration of the pharmaceutical compositions described herein to the patients, as well as use of the compositions described herein in the prevention and treatment of endotoxemia.

The invention provides several advantages. The discoveries described herein with respect to formulation yield a drug product having increased stability, without any sacrifice in drug quality. For example, by including antioxidants, the compositions of the invention are stable to oxidative degradation. In addition, the inclusion of disaccharides and the use of only low amounts of sodium ions enables the maintenance of micelle size throughout the freeze-drying process. Further, the freeze-drying process employed in making the compositions of the invention includes the use of a relatively high shelf temperature, which results in a more efficient formulation process.

Other features and advantages of the invention will be apparent from the following detailed description, the drawings, and the claims.

### BRIEF DESCRIPTION OF THE DRAWINGS

FIG. 1 is a graph showing the relative stability of E5564 in aqueous solution prepared with different drug substance lots, as measured by the formation of major oxidative degradants. The graph shows the HPLC peak area percent of major oxidative degradants present over time for four drug substance lots: 1 (♦), 2 (▪), 3 (▴), and 4 (●), the latter of which includes 7 μg BHA/vial.

FIG. 2 is a flowchart showing a manufacturing scheme for E5564.

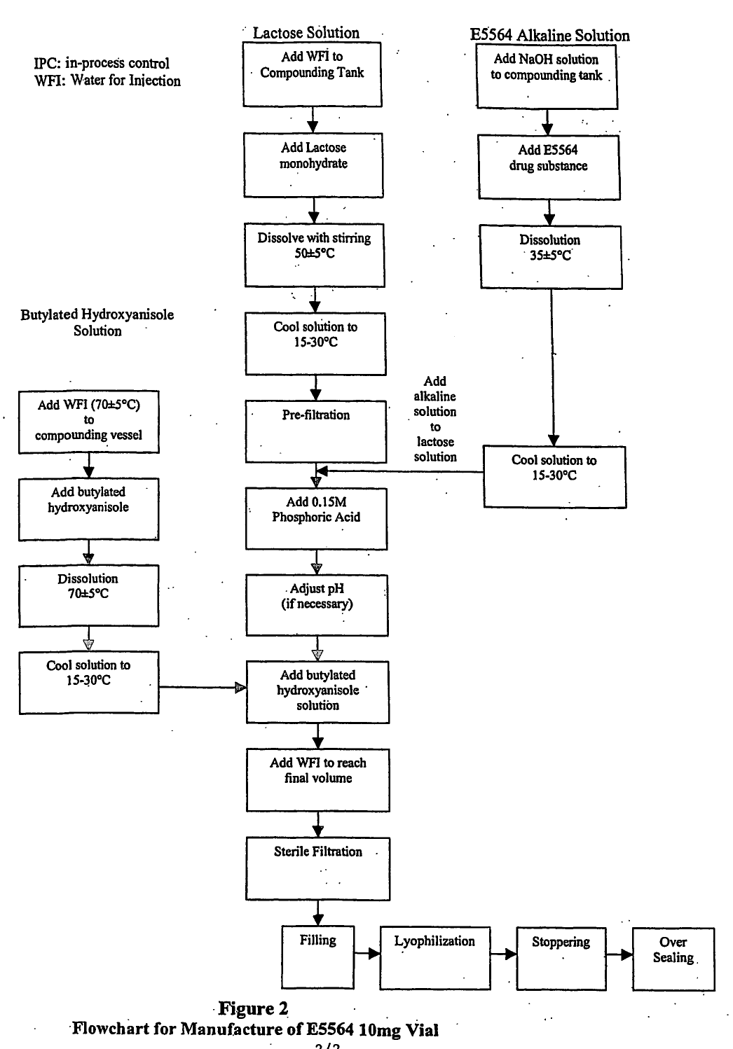

### DETAILED DESCRIPTION

The invention provides pharmaceutical compositions that include an antiendotoxin compound, as well as methods of preparing and using such compositions. The invention is based on the discovery that certain formulation components and steps are particularly advantageous in terms of the quality of the drug product and/or the efficiency of the formulation process. The details of the pharmaceutical compositions of the invention, as well as methods of their production and use, are provided below.

An example of an antiendotoxin compound that can be included in the compositions of the invention is E5564, which has the formula: 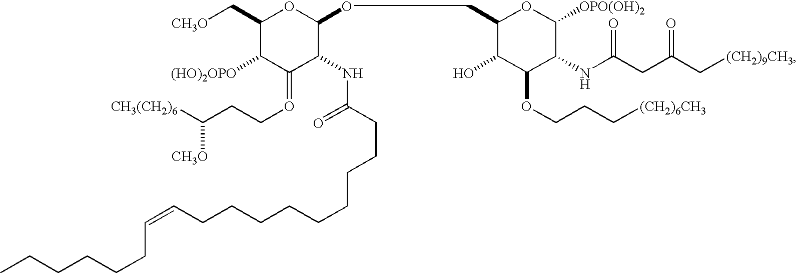
pharmaceutically acceptable salts of this compound. E5564 can be made by using, for example, the synthetic methods described in U.S. Pat. No. 5,935,938, and may be subjected to further purification steps, for example, the purification methods described in international application PCT/US02/16203 (WO 02/094019 A1). Additional examples of antiendotoxin compounds that can be included in the compositions of the invention include compound B531 (U.S. Pat. No. 5,530,113), as well as other antiendotoxin compounds described in these patents and the following U.S. patents: U.S. Pat. No. 5,612,476, U.S. Pat. No. 5,756,718, U.S. Pat. No. 5,843,918, U.S. Pat. No. 5,750,664, and U.S. Pat. No. 5,681,824, the teachings of which are incorporated herein by reference.

The pharmaceutical compositions of the invention can also include, in addition to an antiendotoxin compound, components that we have discovered as providing beneficial features to the compositions. For example, the compositions of the invention can include an antioxidant compound, as we have found that such compounds render the drug product solution stable to degradation by oxidation, without having any adverse effects on drug product quality. An example of an antioxidant compound that can be included in the pharmaceutical compositions of the invention is butylated hydroxyanisole (BHA). Additional examples of antioxidant compounds that can be included in the compositions of the invention are butylated hydroxytoluene (BHT), propyl gallate, sodium sulfite, sodium thiosulfate, monothioglycerol, tert-butyl hydroquinone, ethoxyquin, dithiothreitol, and other antioxidant compounds that are known in the art. Appropriate amounts of these compounds to be included in the compositions of the invention can readily be determined by those of skill in this art, using as guidance, for example, the teachings herein. For example, BHA can be present in the compositions of the invention in amounts ranging from, for example, 0.5-100, 1-50, 2-25, or 5-15 μg/10 mg drug. As a specific example, we note that 7.2 μg BHA is used in a formulation of 10 mg of E5564 that is described in further detail below.

As is discussed further below, we have also found that including a dissacharide, such as lactose, in the compositions of the invention improves the quality of these compositions. In particular, amphiphilic molecules, such as E5564, self associate into micelles in aqueous solution. We have found that including a disaccharide in the compositions of the invention stabilizes the size of the E5564 micelles during lyophilization, as is described in further detail below. Use of disaccharides in the drug formulation process thus facilitates consistency in this process. In addition to lactose, other disaccharides can be included in the compositions of the invention. For example, sucrose, trehalose, or maltose can be used. These compounds can be present in amounts determined to be appropriate by those of skill in this art. For example, they can be present in amounts ranging from 1-20% or 5-15% weight/volume. As a specific example, we note that a formulation of 10 mg of E5564 that is described in further detail below includes 9.7% lactose. Use of this amount provides for good maintenance of micelle size.

We have also found that the ionic strength of the drug solution impacts the stability of the micelle size of the drug during lyophilization. In particular, we have found that minimizing the amount of sodium ions in the drug solution leads to greater stability of the micelle size. In previous methods, sodium phosphate salts had been used to lower the pH of the alkaline solution in which the drug is initially dissolved (see below). We have found that use of phosphoric acid for this purpose can minimize the amount of sodium ions in the formulation, and result in a more stable product. The formulations of the invention can thus include 1-15, e.g., 2-10, mM Na⁺ (or K⁺) (excluding consideration of Na⁺ contributed from the drug). Thus, the compositions of the invention can include, for example, 10 mM Na⁺ (or K⁺) or less, such as, for example, 5, 4, 3, 2, or 1 mM Na⁺ (excluding Na⁺ from the drug)(or K⁺). As a specific example, we note that use of 2 mM Na⁺ (excluding consideration of Na⁺ contributed from the drug) in formulating E5564 results in good stability of micelle size, as is described in further detail below.

Also included in the invention is our discovery of a freeze-drying approach that facilitates more efficient formulation of the drug product, while not adversely affecting product quality. As is discussed further below, the compositions of the invention can be made by using a process including the following steps. First, the drug is dissolved in a dilute, aqueous NaOH solution at pH 10.1-11.8, which facilitates dissolution and dispersion of E5564 into micelles of uniform size. The alkaline E5564 solution is then combined with a lactose solution and a solution including an antioxidant. A phosphoric acid solution is used to neutralize the solution to a pH of about 7.0-8.0. The solution is then adjusted to a target volume with water, filter sterilized, aseptically filled into glass vials, and freeze-dried to render the product stable for long-term storage. As is well known in the art, low shelf temperatures (e.g., −25° C.) are typically used to keep product temperature low, to avoid product collapse during freeze-drying (see, e.g., Pikal, Intl. J. Pharm. 62:165 186, 1990). Surprisingly, we have found that use of relatively high shelf temperatures (e.g., +20° C.) results in good quality product (no collapse). Thus, the invention includes the use of relatively high shelf temperatures (e.g., 0° C.-20° C.) in the freeze-drying process.

The invention also includes methods of making pharmaceutical compositions that include an antiendotoxin compound and an antioxidant, as described herein. These methods include the steps outlined above, i.e, dissolving the drug in a basic solution (e.g., NaOH), addition of a disaccharide stabilizer, addition of an antioxidant (e.g., BHA or any of the other antioxidants listed above), addition of an acidic solution (e.g., phosphoric acid) to lower the pH to 7-8, filtration, and freeze-drying. Detailed examples of each of these steps are provided below. Additional appropriate variations of particular steps in the formulation of E5564 can readily be determined by those of skill in this art (see, e.g., Remington's Pharmaceutical Sciences (18^{th} edition), ed. A. Gennaro, 1990, Mack Publishing Company, Easton, Pa.).

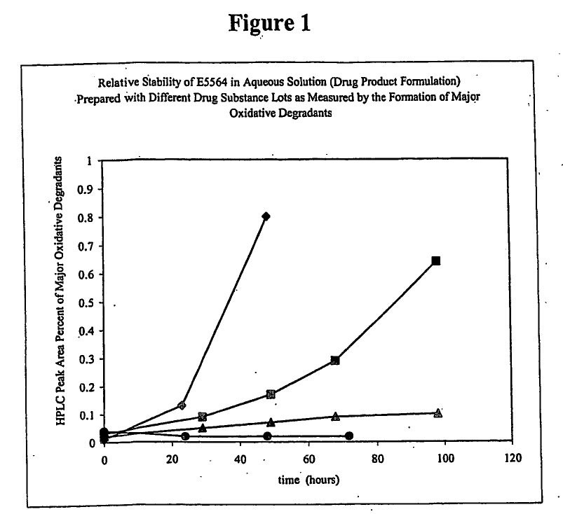

The following is a detailed description of an example of a method for formulating E5564. As is discussed further below, key features of this method include the following. A small amount (7 μg/vial) of butylated hydroxyanisole (BHA) was added to the formulation to prevent oxidation of the E5564 in aqueous solution. As is shown in FIG. 1, inclusion of BHA in the drug product formulation increases its stability in solution. In addition, the sodium content of the solution was lowered by replacing sodium phosphate salts with phosphoric acid for pH adjustment, to enhance micelle size stability during the freeze-drying manufacturing step, and a high shelf temperature was used in the freeze-drying step of the process.

The table below shows the components and composition of an E5564 10 mg vial that is manufactured using the steps set forth below. Also shown in the table is an indication of the function of each component.

Composition of Dosage Form

Component and

| Quality Standard | Strength (Label claim): | | |
|---|---|---|---|
| (and Grade, | 10 mg vial | | Function of |
| if applicable) | Quantity per unit | % | component |
| E5564 Drug | 10.0 mg as free acid, | 0.26% | Active |
| Substance, | 10.7 mg as | | ingredient |
| Eisai Standard | tetrasodium salt | | |
| Lactose | 400 mg | 9.7% | Bulking |
| Monohydrate, NF | | | agent |
| Phosphoric | 0.98 mg | 0.024% | pH |
| Acid, NF ^{A} | | | adjustment |
| Butylated | 0.0072 mg | 0.00017% | Antioxidant |
| hydroxyanisole, | | | |
| NF ^{C} | | | |
| Sodium Hydroxide, | 0.30 mg | 0.0073% | pH |
| NF ^{A} | | | adjustment |
| Water for | 3724 mg | 90.0% | Solvent |
| Injection, USP ^{B} | | | |

^{A} Used for pH adjustment, quantity will vary with lot.
^{B} Water for Injection (WFI) is removed during freeze-drying. Quantity will vary with lot.
^{C} A 10% overage is used to account for manufacturing losses.

Description of a Representative Manufacturing Process
Formulation Compounding
1) Prepare a 5 mM NaOH solution
2) Prepare drug solution by accurately weighing and dissolving E5564 drug substance in NaOH solution at 20° C.-60° C. The pH of the E5564/alkaline solution is pH 10.1-12.0 after dissolution of E5564.
3) Prepare a 0.15M phosphoric acid solution.
4) Preparation a BHA solution.
5) Prepare a lactose solution.
6) Mix the lactose and the drug solutions. Add the phosphoric acid and the BHA solutions and add water to yield the target formulation concentrations.
   Filtration

Filter the formulated solution using a Pall Kleenpak Ultipor® N66® nylon filter with a pore size of 0.2 μm.

Filling and Semi-Plugging

Fill the formulated solution into vials and partially seat lyophilization closures in the vials. Transfer the vials to a freeze-dryer.

Freeze-Drying

Freeze-dry the filled vials under the following conditions:

1) Vials are loaded at +20° C. and then the shelf temperature is lowered to ≦−40° C.
2) The product is held at −40° C. for 3 hours after reaching steady state.
3) Primary drying occurs at shelf temperatures of +20° C. (see below for use of other shelf-temperatures).
4) Secondary drying occurs at shelf temperatures of +20 to +25° C.
5) Pre-aerate the chamber with nitrogen or air.
6) Fully seat the lyophilization closures.
   Sealing

Seal the Vials with Aluminum Caps.

The following section describes experiments carried out to determine the impact of certain variations in formulation parameters on the quality of product formation.

Method of Freeze-Drying E5564 Drug Product

We discovered that a relatively high shelf temperature (e.g., +20° C.) could be used during primary drying to freeze-dry the lactose-containing E5564 drug product. This results in a more efficient manufacturing process, as compared to a conventional low shelf temperature (e.g., −25° C.). Low shelf-temperatures are typically used to keep product temperatures low, to avoid product collapse during freeze-drying (see, e.g., Pikal, Intl. J. Pharm. 62:165-186, 1990). Product collapsed when freeze-dried with Cycle B (see table below), which used a linear increase in shelf temperature from 40° C. to +20° C. at +3° C./hour. It was surprising to discover that Cycles C and D resulted in a good quality product (no collapse), as the shelf temperatures in these cycles were greater than that of Cycle B. Based on these results, we employ a shelf-temperature of 0 to +20° C. with a chamber pressure of <0.1 mmHg. In addition, chamber pressure can be maintained at <0.075 mmHg.

| Cycle | ST (° C.) | P (mmHg) | PT (° C.) | Collapse |
|---|---|---|---|---|
| A | −25 | 0.1 | −31 | no |
| B | −40 to +20 | 0.1 | −31 to −24 | yes |
| C | +20 | 0.1 | −27 | no |
| D | +20 | 0.02 | −28 | no |

ST = shelf temperature
P = chamber pressure
PT = product temperature

Importance of Disaccharides to Formulation

We discovered that disaccharides are useful for preparing freeze-dried preparations of the drug. We have shown that lactose and sucrose are effective at stabilizing the micelle size during freeze-drying (see data in the table set forth below). The micelle size before freeze-drying was 7 nm.

Stabilization of Micelle Size During Lyophilization of E5564 as a Function of Lactose Concentration

| | Micelle Size |
|---|---|
| %(w/v) Lactose | (hydrodynamic |
| in Formulation^{A} | diameter) |
| 1% | 16 nm |
| 2% | 13 nm |
| 5% | 8 nm |
| 10% | 7 nm |

^{A} The micelle size before freeze-drying was 7 nm

Importance of Low Ionic Strength to Formulation

We discovered that minimization of the salt concentration in the formulation is important in maintaining the desired micelle size during freeze-drying. A formulation containing 10 mM Na⁺ (excluding the sodium contributed from the drug) works for some, but not all, lots of E5564. Thus, we employ formulations including 2 mM Na⁺ (excluding the Na⁺ contributed from the drug). Similar parameters apply with respect to potassium ion (K⁺) concentrations.

Importance of Antioxidant to Formulation

We tested several antioxidant compounds to determine whether they impact the stability of E5564 to free-radical oxidation. The table set forth below summarizes the antioxidants tested. We have also found dithiothreitol to be effective.

Antioxidant Screening Experiments

| | | | Major | E5564 | |
|---|---|---|---|---|---|
| Antioxi- | | | Oxidative | conc. (% | |
| dant | time | | Degradant | initial) | Comments |
| none | 0 | hours | 1.12 | 100 | Control |
| none | 18-22 | hours | 6.02 | 91.3 | Rapid degradation |
| BHT | 0 | hours | 0.77 | 100 | |
| BHT | 18-22 | hours | 0.24 | 102.8 | Effective |
| BHA | 0 | hours | 0.68 | 100 | |
| BHA | 18-22 | hours | 0.27 | 102.3 | Effective |
| Propyl | 0 | hours | 0.38 | 100 | |
| Gallate | | | | | |
| Propyl | 18-22 | hours | 0.32 | 100.6 | Effective |
| Gallate | | | | | |
| Vitamin E | 0-4 | hours | 1.34 | 100 | |
| Acetate | | | | | |
| Vitamin E | 18-22 | hours | 4.55 | 94.2 | Not effective |
| Acetate | | | | | |
| Ascorbic | 0-4 | hours | 0.08 | 100 | |
| Acid | | | | | |
| Ascorbic | 18-22 | hours | 0.11 | 97.4 | Not useful, sta- |
| Acid | | | | | bilizing to oxi- |
| | | | | | dation but E5564- |
| | | | | | Ascorbic acid |
| | | | | | react forming new |
| | | | | | impurities |
| Ascorbyl | 0-4 | hours | 0.15 | 100 | |
| Palmitate | | | | | |
| Ascorbyl | 18-22 | hours | 0.19 | 92.4 | Not useful, sta- |
| Palmitate | | | | | bilizing to oxi- |
| | | | | | dation but E5564- |
| | | | | | Ascorbyl palmitate |
| | | | | | react forming new |
| | | | | | impurities |
| Sodium | 0-4 | hours | 0.13 | 100 | |
| Sulfite | | | | | |
| Sodium | 18-22 | hours | 0.25 | 101.8 | Effective |
| Sulfite | | | | | |
| Sodium | 0-4 | hours | 0.09 | 100 | |
| Thiosul- | | | | | |
| fate | | | | | |
| Sodium | 18-22 | hours | 0.19 | 100.2 | Effective |
| Thiosul- | | | | | |
| fate | | | | | |
| Monothio- | 0-4 | hours | 0.13 | 100 | |
| glycerol | | | | | |
| Monothio- | 18-22 | hours | 0.16 | 101.3 | Effective |
| glycerol | | | | | |

Use of the Compositions of the Invention

The compositions of the invention can be used to prevent or to treat any of a large number of diseases and conditions associated with sepsis, septic shock, or endotoxemia. For example, the compositions and methods of the invention can be used in conjunction with any type of surgery or medical procedure, when appropriate, that could lead to the occurrence of endotoxemia or related complications (e.g., sepsis syndrome). As a specific example, the invention can be used in conjunction with cardiac surgery (e.g., coronary artery bypass graft, cardiopulmonary bypass, and/or valve replacement), transplantation (of, e.g., liver, heart, kidney, or bone marrow), cancer surgery (e.g., removal of a tumor), or any abdominal surgery (see, e.g., PCT/US01/01273).

Additional examples of surgical procedures with which the compositions and methods of the invention can be used, when appropriate, are surgery for treating acute pancreatitis, inflammatory bowel disease, placement of a transjugular intrahepatic portosystemic stent shunt, hepatic resection, burn wound revision, and burn wound escharectomy. The compositions of the invention can also be used in conjunction with non-surgical procedures in which the gastrointestinal tract is compromised. For example, the compositions can be used in association with chemotherapy or radiation therapy in the treatment of cancer. The compositions and methods of the invention can also be used in the treatment of conditions associated with HIV infection, trauma, or respiratory distress syndrome, as well as with immunological disorders, such as graft-versus-host disease or allograft rejection. Pulmonary bacterial infection and pulmonary symptomatic exposure to endotoxin can also be treated using the compositions and methods of the invention (see, e.g., PCT/US00/02173).

Administration of the compositions of the invention can be carried out using any of several standard methods including, for example, continuous infusion, bolus injection, intermittent infusion, inhalation, or combinations of these methods. For example, one mode of administration that can be used involves continuous intravenous infusion. In such an approach, the infusion dosage rate of the drug can be, for example, 0.001-0.5 mg/kg body weight/hour, more preferably 0.01-0.2 mg/kg/hour, and most preferably 0.03-0.1 mg/kg/hour, with the drug being infused over the course of, for example, 12-100, 60-80, or about 96 hours. The infusion of the drug can, if desired, be preceded by a bolus injection; preferably, such a bolus injection is given at a dosage of 0.001-0.5 mg/kg. Preferably, the total amount of drug administered to a patient is 25-600 mg of drug, more preferably 35-125 mg, by infusion over a period of 60-100 hours. As activity in the hospital, and particularly the ICU, is often hectic, minor variations in the time period of infusion of the drugs may occur and are also included in the invention.

Additional modes of administration of E5564, according to the methods of the invention, include bolus or intermittent infusion. For example, the drug can be administered in a single bolus by intravenous infusion through, for example, a central access line or a peripheral venous line, or by direct injection, using a syringe. Such administration may be desirable if a patient is only at short-term risk for exposure to endotoxin, and thus does not need prolonged persistence of the drug. For example, this mode of administration may be desirable in surgical patients, if appropriate, such as patients having cardiac surgery, e.g., coronary artery bypass graft surgery and/or valve replacement surgery. In these patients, a single bolus infusion of, e.g., 0.10-15 mg/hour (e.g., 1-7 mg/hour or 3 mg/hour) of drug can be administered over a period of four hours prior to and/or during surgery. (Note that the amount of drug administered is based on an assumed average weight of a patient of 70 kg.) Shorter or longer time periods of administration can be used, as determined to be appropriate by one of skill in this art.

In cases in which longer-term persistence of active drug is desirable, for example, in the treatment of a condition associated with long-term exposure to endotoxin, such as during infection or sepsis, or in appropriate surgical situations in which it is determined that prolonged treatment is desirable, intermittent administration can be carried out. In these methods, a loading dose is administered, followed by either (i) a second loading dose and a maintenance dose (or doses), or (ii) a maintenance dose or doses, without a second loading dose, as determined to be appropriate by one of skill in this art.

The first (or only) loading dose can be administered in a manner similar to that described for the single bolus infusion described above. That is, for E5564 administration, 0.10-15 mg/hour (e.g., 3-7 mg/hour or 3 mg/hour) of drug can be administered to a patient over a period of four hours prior to surgery. If a second loading dosage is to be used, it can be administered about 12 hours after the initial loading dose and can involve infusion of, e.g., 0.10-15 mg/hour (e.g., 1-7 mg/hour or 3 mg/hour) of drug over a period of, e.g., about two hours.

To achieve further persistence of active drug, a maintenance dose (or doses) of drug can be administered, so that levels of active drug are maintained in the blood of a patient. Maintenance doses can be administered at levels that are less than the loading dose(s), for example, at a level that is about ⅙ of the loading dose. Specific amounts to be administered in maintenance doses can be determined by a medical professional, with the goal that drug level is at least maintained. Maintenance doses can be administered, for example, for about 2 hours every 12 hours beginning at hour 24 and continuing at, for example, hours 36, 48, 60, 72, 84, 96, 108, and 120. Of course, maintenance doses can be stopped at any point during this time frame, as determined to be appropriate by a medical professional.

The infusion methods described above can be carried out using catheters (e.g., peripheral venous, central venous, or pulmonary artery catheters) and related products (e.g., infusion pumps and tubing) that are widely available in the art. One criterion that is important to consider in selecting a catheter and/or tubing to use in these methods is the impact of the material of these products (or coatings on these products) on the micelle size of the drug. In particular, we have found that the use of certain products generally maintains a drug micelle size of 7-9 nm. Examples of such catheters are the following Baxter (Edwards) catheters: Swan-Ganz, VANTEX, Multi Med, and AVA Device. Additional examples of catheters that can be used for this purpose are the Becton-Dickinson Criticath catheter; the Arrow International multi-lumen, Arrowg+ard Blue, and Large-bore catheters; and the Johnson & Johnson Protectiv I.V. catheter.

Additional catheter-related products that can be used in the methods of the invention can be identified by determining whether the material of the products alters micelle size of the drug, under conditions consistent with those that are used in drug administration. In addition, in the event that a patient already has a catheter in place that does not maintain optimal drug micelle size, a catheter insert that is made of a compatible material (e.g., a polyamide polymer) or that includes a compatible coating can be used so that the drug solution does not contact the surface of the incompatible catheter. Such an insert, having an outside diameter that is small enough to enable it to be easily inserted into the existing catheter, while maintaining an inside diameter that is large enough to accommodate drug solution flow, is placed within the existing catheter and connected to tubing or a syringe through which the drug is delivered.

In the case of pulmonary bacterial infection or pulmonary symptomatic exposure to endotoxin, administration of the compositions of the invention can be effected by means of periodic bolus administration, by continuous, metered inhalation, or by a combination of the two. A single dose is administered by inhalation 1 μg-24 mg, for example, 5-150 μg, or, preferably, 10-100 μg of the drug. Of course, recalcitrant disease may require administration of relatively high doses, e.g., 5 mg, the appropriate amounts of which can be determined by one of skill in this art. Appropriate frequency of administration can be determined by one of skill in this art and can be, for example, 1-4, for example, 2-3, times each day. Preferably, the drug is administered once each day. In the case of acute administration, treatment is typically carried out for periods of hours or days, while chronic treatment can be carried out for weeks, months, or even years.

Both chronic and acute administration can employ standard pulmonary drug administration formulations, which can be made from the formulations described elsewhere herein. Administration by this route offers several advantages, for example, rapid onset of action by administering the drug to the desired site of action, at higher local concentrations. Pulmonary drug formulations are generally categorized as nebulized (see, e.g., Flament et al., Drug Development and Industrial Pharmacy 21(20):2263-2285, 1995) and aerosolized (Sciarra, “Aerosols,” Chapter 92 in Remington's Pharmaceutical Sciences, 16^{th} edition (ed. A. Osol), pp. 1614-1628; Malcolmson et al., PSTT 1(9):394-398, 1998, and Newman et al., “Development of New Inhalers for Aerosol Therapy,” in Proceedings of the Second International Conference on the Pharmaceutical Aerosol, pp. 1-20) formulations.

All patents and publications mentioned herein are incorporated by reference.

Other embodiments are within the following claims.

What is claimed is:

1. A composition comprising a compound having the formula: 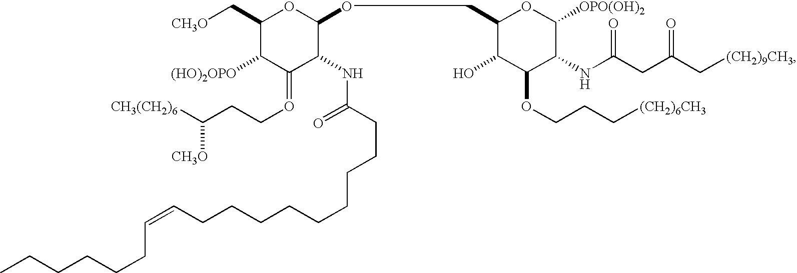
pharmaceutically acceptable salt thereof, and an antioxidant.

2. The composition of claim 1, wherein said antioxidant is selected from the group consisting of butylated hydroxyanisole, butylated hydroxytoluene, propyl gallate, sodium sulfite, sodium thiosulfate, monothioglycerol, tert-butyl hydroquinone, ethoxyquin, dithiothreitol, and derivatives thereof.

3. The composition of claim 1, wherein said antioxidant is butylated hydroxyanisole.

4. The composition of claim 1, further comprising a disaccharide stabilizing agent.

5. The composition of claim 4, wherein said disaccharide is lactose.

6. The composition of claim 4, wherein said disaccharide is sucrose.

7. The composition of claim 1, wherein said composition comprises sodium ions in an amount of 0.5-10 mM.

8. The composition of claim 1, wherein said composition comprises sodium ions in an amount of ≦2 mM.

9. The composition of claim 1, wherein the micelle size of said compound is about 7-9 nm.

10. A method of making a pharmaceutical composition comprising an antiendotoxin compound, said method comprising admixing said compound and an antioxidant.

11. The method of claim 10, wherein said antiendotoxin compound is a compound having the formula: 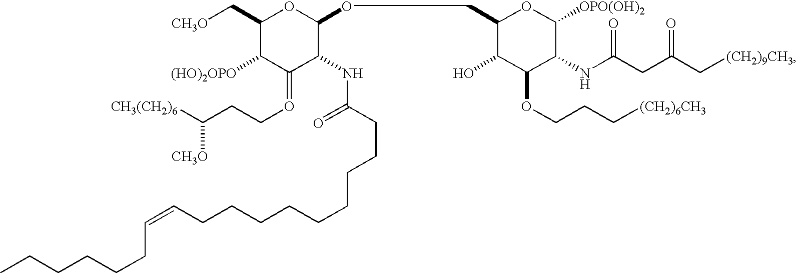
pharmaceutically acceptable salt thereof.

12. The method of claim 10, wherein said antioxidant is selected from the group consisting of butylated hydroxyanisole, butylated hydroxytoluene, propyl gallate, sodium sulfite, sodium thiosulfate, monothioglycerol, tert-butyl hydroquinone, ethoxyquin, dithiothreitol, and derivatives thereof.

13. The method of claim 10, wherein said antioxidant is butylated hydroxyanisole.

14. A method of making a pharmaceutical composition comprising an antiendotoxin compound, said method comprising the steps of:
(i) dissolving said antiendotoxin compound in an aqueous solution of sodium hydroxide;
(ii) adding a disaccharide stabilizer to said solution;
(iii) adding an antioxidant to said solution;
(iv) lowering the pH of said solution;
(v) filter sterilizing said solution; and
(vi) freeze-drying said solution.

15. The method of claim 14, wherein said disaccharide is lactose.

16. The method of claim 14, wherein said disaccharide is sucrose.

17. The method of claim 14, wherein said antioxidant is selected from the group consisting of butylated hydroxyanisole, butylated hydroxytoluene, propyl gallate, sodium sulfite, sodium thiosulfate, monothioglycerol, tert-butyl hydroquinone, ethoxyquin, dithiothreitol, and derivatives thereof.

18. The method of claim 14, wherein said antioxidant is butylated hydroxyanisole.

19. The method of claim 14, wherein said pH of said solution is lowered to about pH 7-8, using a phosphoric acid solution.

20. The method of claim 14, wherein said freeze drying step comprises the use of a shelf temperature of 0° C.-20° C.

21. A method of preventing or treating endotoxemia in a patient, said method comprising administering to said patient the composition of claim 1.

22. Use of the composition of claim 1 in the prevention or treatment of endotoxemia.
